(19) United States
(12) Patent Application Publication
(10) Pub. No.: US 2020/0335224 A1
(43) Pub. Date: Oct. 22, 2020

(54) METHOD AND SYSTEM FOR EVALUATING COMPLIANCE OF STANDARD CLINICAL GUIDELINES IN MEDICAL TREATMENTS

(71) Applicant: KONINKLIJKE PHILIPS N.V., EINDHOVEN (NL)

(72) Inventors: Rithesh SREENIVASAN, Bangalore (IN); Pravin PAWAR, Bangalore (IN); Nagaraju BUSSA, Bangalore (IN); Vikram BASAWARAJ PATIL OKALY, Bangalore (IN); Shiva Moorthy Pookala Vittal BHAT, Bangalore (IN); Rose RAMASAMY, Bangalore (IN); Sudarshan UPADHYA, Bangalore (IN)

(21) Appl. No.: 16/954,568

(22) PCT Filed: Dec. 11, 2018
(86) PCT No.: PCT/EP2018/084258

§ 371 Date: Jun. 17, 2020

(30) Foreign Application Priority Data
Dec. 19, 2017 (EP) 17208488.1

(51) Int. Cl. G16H 50/70; G16H 40/20; G06Q 30/00; G16H 10/60; G16H 70/20; G16H 50/20; G16H 50/50; G16H 20/00; G06F 16/2455; G06F 16/28

(52) U.S. Cl. CPC G16H 50/70; G16H 40/20; G06Q 30/018; G16H 10/60; G06F 16/285; G16H 50/20; G16H 50/50; G16H 20/00; G06F 16/24556; G16H 70/20

(57) ABSTRACT

Disclosed herein is a method and system for evaluating compliance of standard clinical guidelines in medical treatments of one or more patients. Electronic Health Records (EHRs) of the patients are received and aggregated to obtain patient-specific EHRs. Subsequently, the patients are clustered into groups based on disease conditions and socio-demographic characteristics of patients. Further, deviations in the medical treatments provided to the patients are detected by comparing the medical treatments with standard clinical guidelines, for evaluating compliance of the standard clinical guidelines with the medical treatments. In an embodiment, the method of present disclosure helps in detecting care gaps among the medical treatments provided to the patients, and thereby helps in providing improvised and/or patient-specific medical assistance to the patients. Also, the present method is effective in recommending specific amendments and/or improvements to the standard clinical guidelines by detecting nature of deviations in the medical treatments given to the patients.

### FIELD OF THE INVENTION

The present subject matter is related, in general, to compliance monitoring and recommendation system and more particularly, but not exclusively, to a method and system for evaluating compliance of standard clinical guidelines in medical treatments of one or more patients.

### BACKGROUND OF THE INVENTION

Clinical guidelines are systematically developed statements that comprise recommendations and strategies to assist practitioners in making right decisions about appropriate health care in specific clinical circumstances. The clinical guidelines provide guidance in identifying a clinical situation/condition, diagnosing the clinical condition, and/or managing a clinical process associated with a predefined clinical situation. The use of clinical guidelines has a potential to reduce morbidity and mortality rates of patients, while improving the quality of life. Presently, most widely used formats of the clinical guidelines are ‘Computer Interpretable Guidelines (CIGs), which are formal, computer readable versions of the clinical guidelines, consisting of a coordinated composition of medical tasks or actions, with mathematically precise notations and well-defined semantics. The CIGs can be interpreted and manipulated by computers, and allow integration of the clinical guidelines into the clinical information.

Even though the use of clinical guidelines is advised for all the practitioners to treat the patients, many practitioners do not follow the clinical guidelines in the actual practice. It has been seen that many patients with identical clinical problems receive different and conflicting medical treatments depending on factors such as pre-hospital practitioners, ambulance services and hospitals. Further, the nature of medical treatments may also vary depending on various sociodemographic factors such as age, race, ethnicity, language and location of the patients.

A recent study in the domain indicates that, aged patients, living in lowest socio-economic regions experience higher rate of comorbidities compared to the aged patients living in better socio-economic regions. Also, when it comes to the use of clinical guidelines across geographic regions, there may exist multiple clinical guidelines to treat similar patient conditions, depending on the region of the patients and/or research institutions treating the patients, thus making the selection of appropriate clinical guidelines a difficult choice.

In addition, when a patient treatment process becomes successful even without following the clinical guidelines, the treatment processes that resulted in the successful treatment may provide insights into improvements that must be incorporated in the clinical guidelines for that particular patient population. Therefore, it may be essential to draw meaningful insights from retrospective analysis of the patient treatment processes, to make appropriate selection of the clinical guidelines to be used in the treatment of a specific patient population.

### SUMMARY OF THE INVENTION

Disclosed herein is a method for evaluating compliance of standard clinical guidelines in medical treatments of one or more patients. The method comprises receiving, by a health management system, one or more Electronic Health Records (EHRs) related to the one or more patients from one or more data sources. Upon receiving the one or more EHRs, the one or more EHRs corresponding to each of the one or more patients are aggregated into one or more patient-specific EHRs. Further, each of the one or more patients are clustered into one or more patient groups based on similarity in disease conditions and socio-demographic characteristics stored in each of the one or more patient-specific EHRs. Upon clustering each of the one or more patients, deviations in one or more medical treatments of the one or more patients is detected based on comparison of each of the one or more medical treatments, provided to each of the one or more patients in each of the one or more patient groups, with one or more standard clinical guidelines associated with each of the one or more patient groups. Finally, compliance of the one or more standard clinical guidelines in the one or more medical treatments of the one or more patients is evaluated based on the deviations, thus detected.

Further, the present disclosure relates to a health management system for evaluating compliance of standard clinical guidelines in medical treatments of one or more patients. The health management system comprises a processor, and a memory. The memory is communicatively coupled to the processor and stores processor-executable instructions, which on execution, cause the processor to receive one or more Electronic Health Records (EHRs) related to the one or more patients from one or more data sources. Upon receiving the one or more EHRs, the instructions cause the processor to aggregate the one or more EHRs, corresponding to each of the one or more patients, into one or more patient-specific EHRs. Further, the instructions cause the processor to cluster each of the one or more patients into one or more patient groups based on similarity in disease conditions and socio-demographic characteristics stored in each of the one or more patient-specific EHRs. Upon clustering the one or more patients into the one or more groups, the instructions cause the processor to detect deviations in one or more medical treatments of the one or more patients, based on comparison of each of the one or more medical treatments, provided to each of the one or more patients in each of the one or more patient groups, with one or more standard clinical guidelines associated with each of the one or more patient groups. Finally, the instructions cause the processor to evaluate compliance of the one or more standard clinical guidelines in the one or more medical treatments of the one or more patients based on the deviations, thus detected.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The accompanying drawings, which are incorporated in and constitute a part of this disclosure, illustrate exemplary embodiments and, together with the description, explain the disclosed principles. In the figures, the left-most digit(s) of a reference number identifies the figure in which the reference number first appears. The same numbers are used throughout the figures to reference like features and components. Some embodiments of system and/or methods in accordance with embodiments of the present subject matter are now described, by way of example only, and regarding the accompanying figures, in which:

FIG. 1 illustrates an exemplary environment for evaluating compliance of standard clinical guidelines in medical treatments of one or more patients in accordance with some embodiments of the present disclosure;

FIG. 2 shows a detailed block diagram illustrating a health management system for evaluating compliance of standard clinical guidelines in medical treatments of one or more patients in accordance with some embodiments of the present disclosure;

FIG. 3A is an exemplary representation of an Electronic Health Record (EHR) in accordance with some embodiments of the present disclosure;

FIG. 3B illustrates detection of deviations in the one or more medical treatments, during a treatment process, given to the one or more patients in accordance with some embodiments of the present disclosure;

FIG. 4 shows a flowchart illustrating a method for evaluating compliance of standard clinical guidelines in medical treatments of one or more patients in accordance with some embodiments of the present disclosure; and

FIG. 5 illustrates a block diagram of an exemplary computer system for implementing embodiments consistent with the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether such computer or processor is explicitly shown.

### DETAILED DESCRIPTION OF EMBODIMENTS

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

While the disclosure is susceptible to various modifications and alternative forms, specific embodiment thereof has been shown by way of example in the drawings and will be described in detail below. It should be understood, however that it is not intended to limit the disclosure to the specific forms disclosed, but on the contrary, the disclosure is to cover all modifications, equivalents, and alternative falling within the spirit and the scope of the disclosure.

The terms “comprises”, “comprising”, “includes”, or any other variations thereof, are intended to cover a non-exclusive inclusion, such that a setup, device, or method that comprises a list of components or steps does not include only those components or steps but may include other components or steps not expressly listed or inherent to such setup or device or method. In other words, one or more elements in a system or apparatus proceeded by “comprises . . . a” does not, without more constraints, preclude the existence of other elements or additional elements in the system or method.

The present disclosure relates to a method and a health management system for evaluating compliance of standard clinical guidelines in medical treatments of one or more patients. In an embodiment, the method of present disclosure includes federation of multiple Electronic Health Record (EHR) systems, associated with the one or more patients, to keep a track of one or more medical treatments given to the one or more patients. Further, data integration and normalization operations are performed on each of the one or more EHRs corresponding to each of the one or more patients, to obtain a standard, patient-specific EHR.

Subsequently, each of the one or more patients are clustered into multiple patient groups based on health condition and/or disease conditions and socio-demographic characteristics stored in the patient-specific EHRs corresponding to each of the one or more patients. Further, a retrospective analysis of the one or more medical treatments given to each of the one or more patients in each of the one or more patient groups is performed with respect to one or more predetermined standard clinical guidelines, to identify deviations in the one or more medical treatments over a given period of time.

In an embodiment of the present disclosure, the deviations in the one or more medical treatments, identified by the retrospective analysis of the one or more treatments, are further classified as negative deviations and positive deviations to derive further insights into the one or more medical treatments given to each of the one or more patients. In an embodiment, the deviations classified as the negative deviations may be used to determine care gaps in the one or more medical treatments, and relevant gap analysis results/reports may be generated. Further, the gap analysis reports/results may be provided to hospitals or care-takers of the one or more patients to improve their conformance to the standard clinical guidelines, thereby improving quality of one or more treatments given to the one or more patients. Similarly, the one or more medical treatments resulting into the positive deviations may be suggested to the relevant hospitals or practitioners for updating and/or improving the standard clinical guidelines.

In an embodiment, the method of present disclosure may be useful in scenarios where clinical practitioners do not refer to the standard clinical guidelines while treating a patient. Here, the steps taken by the practitioners to treat the patient are mainly based on practitioner's prior experience and judgement of the disease condition of the patients. This may result in a situation where the treatment given to the patient may vary from the treatment, which is actually prescribed in a standard clinical guideline. In such scenarios, the present method helps in detecting the compliance between the treatments received by patients and the treatments prescribed in the standard clinical guidelines, by comparing chronological treatment record from the patient-specific EHRs with the prescribed treatment in the standard clinical guidelines.

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the disclosure, and it is to be understood that other embodiments may be utilized and that changes may be made without departing from the scope of the present disclosure. The following description is, therefore, not to be taken in a limiting sense.

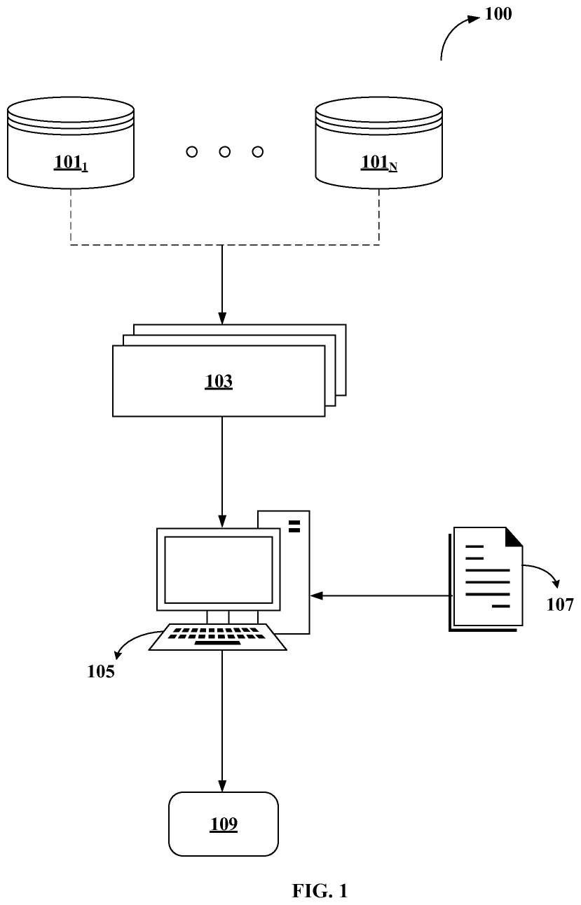

FIG. 1 illustrates an exemplary environment 100 for evaluating compliance of standard clinical guidelines 107 in medical treatments of one or more patients in accordance with some embodiments of the present disclosure.

The environment 100 may include one or more data sources, data source 1 101₁ to data source N 101_{N} (hereinafter collectively referred to as data sources 101), and a health management system 105. The data sources 101 may be associated with one or more hospitals related to one or more patients, and may store one or more Electronic Health Records (EHRs) 103 of the one or more patients. In an embodiment, the one or more EHRs 103 may be electronic versions of medical records of the one or more patients, which are maintained at the one or more hospitals over a period of time. As an example, each of the one or more EHRs 103 may include, without limiting to, medical history of the one or more patients, disease conditions of the one or more patients, and socio-demographic characteristics of the one or more patients. In an embodiment, the one or more EHRs 103 may be constantly updated by the one or more hospitals, such that the one or more EHRs 103 would always include most recent medical profile/status of the one or more patients.

In an embodiment, the health management system 105 may be a computing device that can be configured to receive one or more EHRs 103 related to one or more patients from the data sources 101. As an example, the health management system 105 may receive the one or more EHRs 103 at predetermined regular intervals, for example, once in every 24 hours. Upon receiving the one or more EHRs 103 from the data sources 101, the health management system 105 may perform data normalization operations on each of the one or more EHRs 103 to eliminate one or more inconsistencies from each of the one or more EHRs 103. Thereafter, the health management system 105 may aggregate each of the one or more EHRs 103 into one or more patient-specific EHRs 103, which include information that are specific to each of the one or more patients. In some scenarios, information related to the one or more patients may be stored in more than one EHRs 103 across multiple hospitals and/or locations. In such scenarios, the aggregation process may be useful for collating each such multiple EHRs 103 into a single, patient-specific EHR, and thereby maintaining unique patient-specific EHRs 103 for each of the one or more patients.

In an embodiment, upon generating the one or more patient-specific EHRs 103, corresponding to each of the one or more patients, the health management system 105 may cluster each of the one or more patients into one or more patient groups based on similarity in disease conditions and socio-demographic characteristics stored in each of the one or more patient-specific EHRs 103. As an example, the one or more patients suffering from a common disease/illness such as ‘solid tumor’, may be clustered into a single group.

In an embodiment, after forming the one or more patient groups, the health management system 105 may scan through one or more clinical guidelines repositories (not shown in FIG. 1), associated with the health management system 105, for identifying and retrieving one or more standard clinical guidelines 107 that relate to the one or more patient groups. Relevance between the one or more standard clinical guidelines 107 and the one or more patient groups may be established based on the disease conditions and the socio-demographic characteristics associated with the one or more patient groups. As an example, the standard clinical guideline that may be used for treating one or more patients living in region ‘A’, and belonging to a common patient group, say ‘solid tumor’, may be the standard clinical guideline, which is specifically prescribed for treating the one or more patients of region ‘A’, and suffering from ‘solid tumor’.

Upon clustering each of the one or more patients into one or more patient groups, and associating the one or more standard clinical guidelines 107 to each of the one or more patients in each of the one or more patient groups, the health management system 105 may detect deviations 109 in the one or more medical treatments given for each of the one or more patients across each of the one or more patient groups. In an embodiment, the deviations 109 may be detected based on comparison of each of the one or more medical treatments provided to each of the one or more patients with one or more standard clinical guidelines 107 associated with each of the one or more patient groups.

Finally, compliance of the one or more standard clinical guidelines 107 in the one or more medical treatments of the one or more patients may be evaluated based on the deviations 109 in the one or more medical treatments. In an embodiment, for further evaluation of the deviations 109, the deviations 109 may be classified as positive deviations and negative deviations based on health condition of the one or more patients over a period of treatment. For example, the deviations 109 may be classified as positive deviations, when post-treatment health condition of the one or more patients appears to be improved than pre-treatment health condition of the one or more patients. Similarly, the deviations 109 may be classified as negative deviations, when the post-treatment health condition of the one or more patients appears to have been deteriorated in comparison to the pre-treatment health condition of the one or more patients.

In an embodiment, subsequent to classifying the deviations 109 as the positive deviations and the negative deviations, the health management system 105 may notify a practitioner and/or caretaker, associated with the one or more patients, about nature of the deviations 109. For example, upon detecting the positive deviations in the one or more medical treatments, the health management system 105 may notify the one or more medical treatments that have resulted into the positive deviations to one or more practitioners for recommending one or more amendments and/or improvisations in the standard clinical guidelines 107. Similarly, upon detecting the negative deviations in the one or more medical treatments, the health management system 105 may notify the one or more medical treatments that have resulted into the negative deviations to the one or more practitioners and/or care takers of the one or more patients for suggesting one or more improvisations to be included in the one or more medical treatments given to the one or more patients.

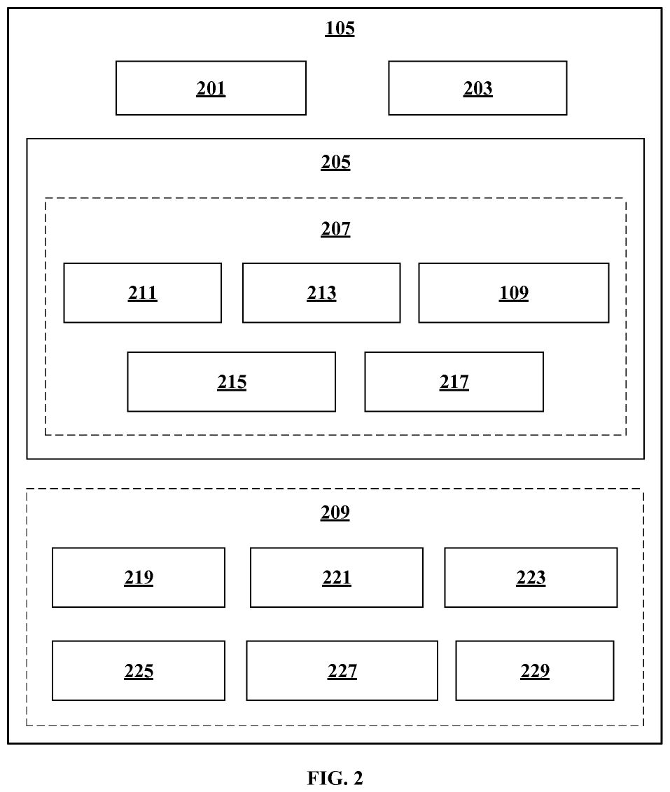

FIG. 2 shows a detailed block diagram illustrating a health management system 105 for evaluating compliance of standard clinical guidelines 107 in medical treatments of one or more patients in accordance with some embodiments of the present disclosure.

In an implementation, the health management system 105 may include an I/O interface 201, a processor 203, and a memory 205. The I/O interface 201 may be configured to communicate with one or more data sources 101 for receiving one or more Electronic Health Records (EHRs) 103 of each of the one or more patients. Further, the I/O interface 201 may be used to connect an end-user device associated with one or more practitioners or caretakers of the one or more patients to notify deviations 109 in the one or more medical treatments of the one or more patients. In an embodiment, the memory 205 may be communicatively coupled to the processor 203. The processor 203 may be configured to perform one or more functions of the health management system 105 for evaluating compliance of standard clinical guidelines 107 in medical treatments of one or more patients.

In some implementations, the health management system 105 may include data 207 and modules 209 for performing various operations in accordance with the embodiments of the present disclosure. In an embodiment, the data 207 may be stored within the memory 205 and may include, without limiting to, patient-specific EHRs 211, patient groups 213, deviations in medical treatments 109 (also referred to as the deviations 109), recommendations 215 and other data 217.

In some embodiments, the data 207 may be stored within the memory 205 in the form of various data structures. Additionally, the data 207 may be organized using data models, such as relational or hierarchical data models. The other data 217 may store data, including temporary data and temporary files, generated by the modules 209 while performing various functions of the health management system 105.

In an embodiment, the one or more patient-specific EHRs 211 are obtained by aggregating each of the one or more EHRs 103, received from the data sources 101. Each of the one or more patient-specific EHRs 211 may store medical information that are specific to each of the one or more patients. As an example, the patient-specific EHRs 211 for a patient ‘A’ may include, without limiting to, information related to key administrative clinical data relevant to the treatments provided to the patient ‘A’ under a particular provider, including socio-demographic characteristics, progress notes, medical problems/illnesses, perceived medications, vital signs, past medical history, immunizations, laboratory data, radiology reports and the like. In an embodiment, the information in each of the one or more patient-specific EHRs 211 may be stored in a predetermined health information exchange standard format such as Fast Health Interoperability Resources (FHIR) standard.

In an embodiment, the patient groups 213 may be formed by clustering each of the one or more patients based on similarity in the disease conditions and socio-demographic characteristics stored in each of the one or more patient-specific EHRs 211. As an example, the one or more patients suffering from an issue of ‘heart failure’ may be clustered into a single group.

In an embodiment, the deviations 109 in the one or more medical treatments may be detected by comparing each of the one or more medical treatments, provided to each of the one or more patients in each of the one or more patient groups 213, with one or more standard clinical guidelines 107 associated with each of the one or more patient groups 213. Further, the deviations 109 in the one or more medical treatments may be positive deviations or negative deviations. In an embodiment, the deviations 109 in the one or more medical treatments may be considered as the positive deviations, when the post-treatment health condition of the one or more patients is improved than the pre-treatment health condition of the one or more patients. In other words, the deviations 109 may be classified as the positive deviations, when the one or more patients seem to have been diagnosed and treated successfully even though the one or more medical treatments given to the one or more patients are not in compliance with the one or more standard clinical guidelines 107. Similarly, the deviations 109 in the one or more medical treatments may be considered as the negative deviations, when the post-treatment health condition of the one or more patients has deteriorated than the pre-treatment health condition of the one or more patients.

In an embodiment, the recommendations 215 may be provided to the one or more practitioners and/or care takers of the one or more patients. The nature of the recommendations 215 may depend on the nature of the deviations 109 in the one or more medical treatments. For example, when the deviations 109 are the positive deviations, the recommendations 215 may include one or more suggestions for amending and/or upgrading the one or more standard clinical guidelines 107 for improvising the one or more standard clinical guidelines 107 with the one or more medical treatments that have resulted into the positive deviations. Similarly, when the deviations 109 are the negative deviations, the recommendations 215 may include one or more suggestions to the one or more practitioners and/or care takers of the one or more patients for changing the one or more medical treatments in a way that eliminates or reduces the care gaps in the one or more medical treatments.

In an embodiment, the data 207 may be processed by one or more modules 209 of the health management system 105. In one implementation, the one or more modules 209 may be stored as a part of the processor 203. In another implementation, the one or more modules 209 may be communicatively coupled to the processor 203 for performing one or more functions of the health management system 105. The modules 209 may include, without limiting to, a receiving module 219, a data aggregation module 221, a clustering module 223, a deviation detection module 225, a deviation evaluation module 227, and other modules 229.

As used herein, the term module refers to an application specific integrated circuit (ASIC), an electronic circuit, a processor (shared, dedicated, or group) and memory that execute one or more software or firmware programs, a combinational logic circuit, and/or other suitable components that provide the described functionality. In an embodiment, the other modules 229 may be used to perform various miscellaneous functionalities of the health management system 105. It will be appreciated that such modules 209 may be represented as a single module or a combination of different modules.

In an embodiment, the receiving module 219 may be used for receiving the one or more EHRs 103 from the data sources 101. The receiving module 219 may receive the one or more EHRs 103 in predetermined regular intervals, such that, at any given time, the health management system 105 must have access to a most recent version of the one or more EHRs 103 for making effective evaluation of the compliance of the one or more treatments against the one or more standard clinical guidelines 107. As an example, the predetermined regular interval may be 24 Hrs. Alternatively, the receiving module 219 may be configured to dynamically receive the one or more EHRs 103, as soon as there has been an update in any information related to the one or more patients. In an implementation, the one or more EHRs 103 may be received in a standard health interoperability format such as the FHIR.

In an embodiment, the data aggregation module 221 may be used for aggregating the one or more EHRs 103 corresponding to the one or more patients into one or more patient-specific EHRs 211. Since it may happen that the one or more EHRs 103 corresponding to a single patient are stored in multiple data sources 101, the data aggregation process ensures that each of the one or more EHRs 103 received from the multiple data sources 101, and belonging to a single patient, are stored as a common patient-specific EHR. During the aggregation process, the data aggregation module 221 may organize each of the one or more EHRs 103 in a chronological order to enable tracking of the patient's treatment history since the beginning of the one or more treatments provided to the one or more patients

In an embodiment, the data aggregation module 221 may be configured to perform a data normalization operation on the information stored in each of the one or more patient-specific EHRs 211 to improve integrity of the information stored in the one or more patient-specific EHRs 211. Various information such as medical profile of the one or more patients, and a unique identifier of the one or more patients, may be stored repetitively in the one or more EHRs 103 received from the multiple data sources 101. Hence, the data normalization operation ensures that all the redundant patient information are removed from the one or more patient-specific EHRs 211, and a consolidated patient information is retained in the health management system 105 for evaluating the one or more treatments provided to the one or more patients.

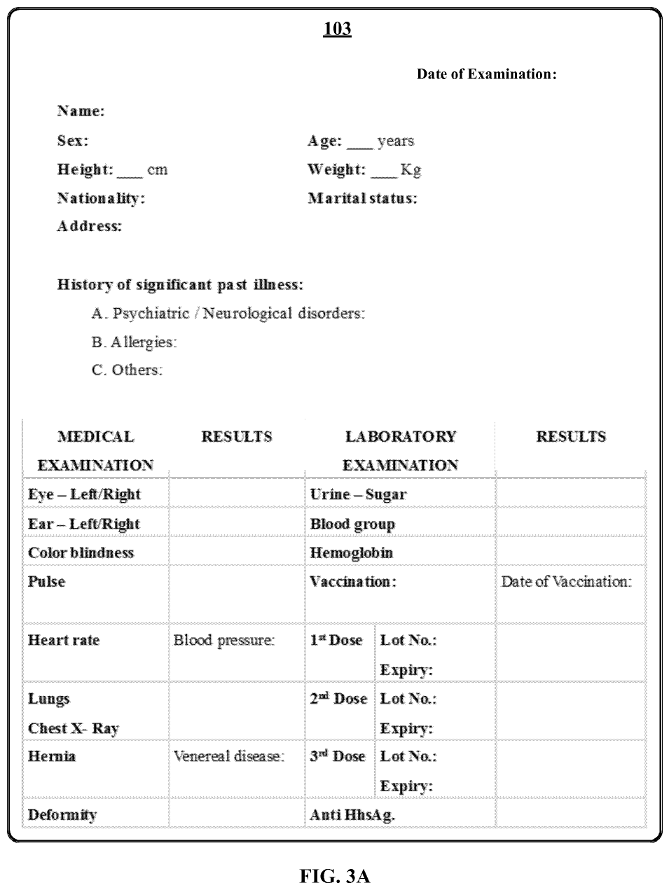

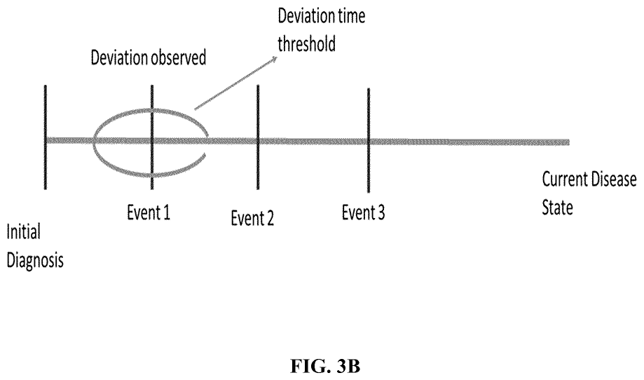

In some implementations, the patient information may be represented using a predetermined medical data representation format such as International Classification of Diseases (ICD)-9, or ICD-10 standard codes depending on nature of applications/implementation. In certain cases, only the names of the disease conditions of the one or more patients might be included without using any ICD standard codes. Thus, the data normalization operation ensures that diagnosis of each of the one or more patients is recorded uniformly using an agreed upon data representation format such as ICD-9 or ICD-10 standard codes. An exemplary representation of the EHR 103 may be as illustrated in FIG. 3A.

In an embodiment, the clustering module 223 may be used for clustering each of the one or more patients into one or more patient groups 213 based on similarity in disease conditions and socio-demographic characteristics stored in each of the one or more patient-specific EHRs 211. The clustering module 223 may perform a chronological evaluation of each of the one or more patient-specific EHRs 211 to identify one or more of diseases suffered by each of the one or more patients. Further, each of the one or more patients may be grouped based on the associated disease combinations, which is also known as comorbidities. In other words, the one or more patients with same/similar combination of the one or more disease conditions may be placed in a single group. Table A below indicates grouping of the one or more patients into five exemplary patient groups 213, represented by Group numbers—1 to 5, based on the similarity in the disease conditions of each of the one or more patients.

TABLE A
Clustering of patients into patient groups

| Group | No. of | |
|---|---|---|
| number. | patients | Disease conditions (comorbidities) |
| 1 | 234 | Heart failure |
| 2 | 126 | Heart failure + Hypertension |
| 3 | 53 | Heart failure + Hypertension + Chronic pulmonary |
| 4 | 156 | Solid tumor |
| 5 | 34 | Solid tumor + Hypertension |

In an embodiment, the deviation detection module 225 may be used for detecting deviations 109 in the one or more medical treatments provided to the one or more patients, based on comparison of each of the one or more medical treatments with one or more standard clinical guidelines 107 associated with each of the one or more patient groups 213. Generally, each of the one or more standard clinical guidelines 107 are organized according to the disease conditions and the socio-demographic characteristics of the one or more patients. For example, as shown in Table B, for a given disease condition, multiple standard clinical guidelines 107 may be proposed based on geographical region/location of the patient, a specific age-group of the patient, gender of the patient, and the similar factors.

TABLE B
Classification of the standard clinical guidelines based on the
Disease conditions and the socio-demographic characteristics.

| Name of Clinical | Targeted Disease | Geographic | | |
|---|---|---|---|---|
| Guideline | condition | region | Age group | Gender |
| ICSI guideline for | Heart failure | USA | Adults | Male and |
| heart failure in adults | | | (>18 yrs.) | Female |
| ACCF/AHA guideline | Heart failure | USA | Adults | Male and |
| for the management of | | | (>18 yrs.) | Female |
| heart failure | | | | |
| ESC Guidelines for the | Acute heart failure; | EU | Adults | Male and |
| diagnosis and | Chronic heart | | (>18 yrs.) | Female |
| treatment of acute and | failure | | | |
| chronic heart failure | | | | |
| SIGN guideline for the | Chronic heart | UK | Adults | Male and |
| Management of | failure | | (>18 yrs.) | Female |
| Chronic Heart Failure | | | | |
| NHF Guideline for the | Hypertension; | Australia | Adults | Male and |
| diagnosis and | Hypertension & | | (>18 yrs.) | female |
| management of | Chronic kidney | | | |
| hypertension in adults | disease; | | | |
| | Hypertension & | | | |
| | Myocardial | | | |
| | infarction; | | | |
| | Hypertension & | | | |
| | Chronic heart | | | |
| | failure; | | | |
| | Hypertension & | | | |
| | Peripheral arterial | | | |
| | disease; | | | |

Thus, in order to effectively detect the deviations 109 in the one or more treatments, the deviation detection module 225 may, as an initial step, identify the one or more standard clinical guidelines 107 that are relevant to the one or more disease conditions of the one or more patients in the one or more patient groups 213. As an example, for a patient group having one or more patients suffering from ‘Heart failure’, the one or more relevant standard clinical guidelines 107 to be identified from the list of standard clinical guidelines 107 (as shown in Table B) may be as shown in Table C.

TABLE C
standard clinical guidelines relevant for
the disease condition - Heart failure.
Targeted Disease condition: Heart failure

| Geographic | | | Relevant Clinical |
|---|---|---|---|
| region | Age group | Gender | guidelines |
| USA | Adults | Male | ICSI guideline, |
| | (>18 yrs.) | | ACCF/AHA guideline |
| USA | Adults | Female | ICSI guideline, |
| | (>18 yrs.) | | ACCF/AHA guideline |
| UK | Adults | Male | SIGN guideline |
| | (>18 yrs.) | | |
| UK | Adults | Female | SIGN guideline |
| | (>18 yrs.) | | |
| Netherlands | Adults | Male | ESC guideline |
| | (>18 yrs.) | | |
| Netherlands | Adults | Female | ESC guideline |
| | (>18 yrs.) | | |

Upon identifying the one or more standard clinical guidelines 107 relevant to each of the one or more patients, the deviation detection module 225 may detect the deviations 109 in the one or more treatments by comparing each of the one or more medical treatment processes, prescribed in the one or more standard clinical guidelines 107, against each of the one or more medical treatments that were actually provided to the one or more patients. The deviations 109 in the one or more treatments may be detected when the one or more treatments provided to the one or more patients do not match with each of the one or more medical treatments prescribed in the one or more standard clinical guidelines 107.

In an embodiment, the deviation detection module 225 may use predetermined conformance detection algorithms to determine conformance between the one or more medical treatments and the one or more standard clinical guidelines 107 for each of the one or more patients across each of the one or more patient groups 213. As an example, the conformance detection algorithm used for determining the conformance may be State-Transition Audit (ST-Audit) based technique, which audits each patient record for conformance to a particular standard clinical guideline. In the ST-Audit technique, each state may have a set of attributes that describe all actions and decisions that must be made during the patient's consultation to the one or more practitioners. The set of attributes may include list of medical exams that must be performed in the state, questions to be asked to the patient, medications to be prescribed to the patient, next appointment and transitions. The transitions help in defining conditions for a patient to move to another state.

Further, a standard clinical guideline may be modelled as a state diagram, in which the states represent stages in the patient treatment/management process, and the transitions among the states may be evaluated based on results of medical examinations and patient responses received for the questions asked to the patient. If a transition is true, then the state of the standard clinical guideline may change to a different state, which represents a different stage in the patient's treatment process. Here, the ST-Audit technique may be used to match a sequence of the patient's consultations at different states in the standard clinical guideline. Further, the ST-Audit technique may be used to determine whether a healthcare professional and/or the practitioner has complied to the standard clinical guidelines 107, whether unnecessary medical tests were suggested, whether medications were prescribed correctly, and the like.

In an embodiment, various other techniques such as intention-based critiquing of clinical guidelines oriented medical care, may be used for determining the conformance, in place of the ST-Audit algorithm. Table D below indicates application of the ST-Audit technique for performing a state-wise compliance check for the standard clinical guideline—‘Sixth Joint National Committee (VI JNC) Guideline for Hypertension’ for the disease condition—‘Hypertension’.

TABLE D
Detection of deviations of the standard clinical guidelines in the medical treatments
Targeted Disease condition: Hypertension
Relevant Clinical Guideline: VI JNC Guideline for Hypertension

| | | | | | Possible |
|---|---|---|---|---|---|
| | | | | Possible | unnecessary/ |
| Visit | | | | unnecessary/ | missing |
| No. | Exams | Medication | Conclusion | missing exams | medication |
| 1 | Sbp: 170 | None | In agreement | None | None |
| | Dbp: 102 | | with guideline | | |
| 2 | Sbp: 174 | Ace inhibitors - | Not in | None | Unnecessary |
| | Dbp: 114 | 50 mg/day, | agreement | | Diuretics |
| | | Diuretics - | with guideline | | |
| | | 1 mg/day | | | |
| 3 | Sbp: 140 | Ace inhibitors - | In agreement | Urinary | None |
| | Dbp: 100 | 50 mg/day, | with guideline | Ultrasonography, | |
| | | Diuretics - | | Urine, | |
| | | 25 mg/day | | Creatinine | |
| 4 | Sbp: 146 | Ace inhibitors - | Not in | None | Low medication |
| | Dbp: 104 | 50 mg/day, | agreement | | dosage |
| | | Diuretics- | with guideline | | |
| | | 25 mg/day, | | | |
| | | Beta-blockers - | | | |
| | | 80 mg/day | | | |
| 5 | Sbp: 128 | Ace inhibitors - | In agreement | None | None |
| | Dbp: 90 | 50 mg/day, | with guideline | | |
| | | Diuretics- | | | |
| | | 25 mg/day, | | | |
| | | Beta-blockers - | | | |
| | | 80 mg/day | | | |

Sbp—Systolic blood pressure;
Dbp—Diastolic blood pressure

In an embodiment, referring to Table D, the deviations 109 detected in one or more stages of the patient treatment process (represented by visit numbers) may be depicted on a medication progression graph, such as the graph shown in FIG. 3B. Here, each stage in the patient treatment process may be indicated on a time-progression line, along with the deviations 109 observed in each stages of the treatment process. The initial stage of treatment or the initial diagnosis stage may be marked from the patient's first visit to the practitioner. Thereafter, one or more stages of the treatment process may be determined at subsequent visits made by the patient. Further, nature and time of occurrence of each deviation 109 in the one or more stages of the treatment process may be listed and used for further evaluation of the deviations 109.

In an embodiment, the deviation evaluation module 227 may be used to evaluate the deviations 109 that are detected in the one or more medical treatments. The deviations 109 may be evaluated by classifying the deviations 109 as the positive deviations and the negative deviations, and then determining significance of each deviations 109 on the post-treatment health condition of the one or more patients. In an embodiment, the deviation evaluation module 227 may quantify the deviations 109 in the one or more treatments based on the following process:
1) Mapping the detected deviations 109 back into a treatment progression timeline of the patient treatment process.
2) Identifying one or more similar patient records (having similar socio-demographic characteristics, disease conditions etc.) to the current patient record, such that, the identified similar patient records do not exhibit deviations 109 from the standard clinical guidelines 107.
3) Extracting the positive outcomes (i.e. positive deviations) for each of the identified similar patient records as ground truth data for evaluation.
4) Comparing the outcome of current patient record, where a deviation is observed, with the positive outcomes of each of the similar patient records based on the ground truth data for quantifying the deviations 109 in the current patient record as the positive deviations or the negative deviations within predetermined threshold time.
5) Computing clinical significance of the deviations 109 based on the similar deviations 109 in a set of patients having similar set of disease conditions and within the same predetermined threshold time following an event in the treatment process. Here, the clinical significance may be computed using standard measures such as disclosed in Edwards-Nunnally, Jacobson-Truax and the like.

In an embodiment, upon quantifying the deviations 109 in the one or more treatments, the deviation evaluation module 227 may determine most probable recommendations 215 for improvements in the one or more standard clinical guidelines 107, and suggestions to handle care gaps in the one or more medical treatments. The compliance results obtained in the above process may be classified as shown in Table E below, for illustrating clinical significance of each category of the deviations 109.

TABLE E
Compliance status and the corresponding significance on
the post-treatment health condition of the patients.

| Compliance to Standard | Post-treatment health |
|---|---|
| clinical guidelines | condition of the patient |
| High | Improved |
| Positive Deviation | Improved |
| Negative Deviation | Deteriorated |
| Low | Deteriorated |

As indicated in Table E, the post-treatment health condition of the patient may be said to be deteriorated when the disease condition is not managed well, and the clinical parameters are out of the expected range. Similarly, the post-treatment health condition of the patient is said to be improved when the disease condition has been managed well, and the patient clinical parameters are within the expected range.

In an embodiment, the compliance categories that are indicated as ‘Negative Deviation’ and low′ may be further analyzed to identify if the deviations 109 are applicable at a local level or global level, in terms of the geographic locations of each of the one or more patients. Accordingly, for each deviation, a hierarchical clustering of the one or more patients is performed based on the socio-demographic characteristics of each of the one or more patients. For example, the parameters that are considered for the hierarchical clustering may include, without limiting to, country of residence, corresponding zip codes, gender and age categories, language, ethnicity, weather conditions, education level and income level of each of the one or more patients. Finally, one or more sub-groups of the patients may be formed as shown Table F below, based on the hierarchical clustering of the patients.

TABLE F
hierarchical clustering of the patients
upon evaluating the deviations.

| Sub-group | No. of | Geographic | | |
|---|---|---|---|---|
| No. | patients | region | Age group | Gender |
| 1 | xxxxx | USA | Adults | Male |
| | | | (>18 yrs.) | |
| 2 | xxxxxx | USA | Adults | Female |
| | | | (>18 yrs.) | |
| 3 | xxxxxx | UK | Adults | Male |
| | | | (>18 yrs.) | |
| 4 | xxxxxx | UK | Adults | Female |
| | | | (>18 yrs.) | |
| 5 | xxxxx | Netherlands | Adults | Male |
| | | | (>18 yrs.) | |
| 6 | xxxxx | Netherlands | Adults | Female |
| | | | (>18 yrs) | |

In an embodiment, when the size of each of the one or more sub-groups (in terms of total number of patients) constitutes a clinically significant number, the deviations 109 corresponding to such sub-groups may be recommended to the one or more practitioners at a local level (i.e. at a local geographical region) for improving the care gaps in that region. Further drill down of care gaps may be done at various levels of the socio-demographic characteristics such as zip codes, socio-economic factors, accessibility etc. for an effective evaluation of the deviations 109 in the one or more treatments. For example, similar analysis of the deviations 109 in the multiple patient sub-groups may be considered to determine if there are similarities between the deviations 109 in similar socio-demographic characteristics. Finally, each the one or more patient sub-groups that exhibit similar deviations 109 may be combined into a global cluster, which can span a larger geographic region. The clusters of patients, thus obtained, may be helpful in deriving clinical insights at higher level, and appropriate reports may be generated and notified to the practitioners and care providers in those regions.

In an embodiment, similar clustering strategies may be used for clustering the one or more patient sub-groups exhibiting positive deviations, and the appropriate reports may be generated and notified to the relevant practitioners for improving their practices. The evaluation of the positive deviations may also be helpful for recommending the same as evidence for making amendments and/or modifications in the one or more standard clinical guidelines 107.

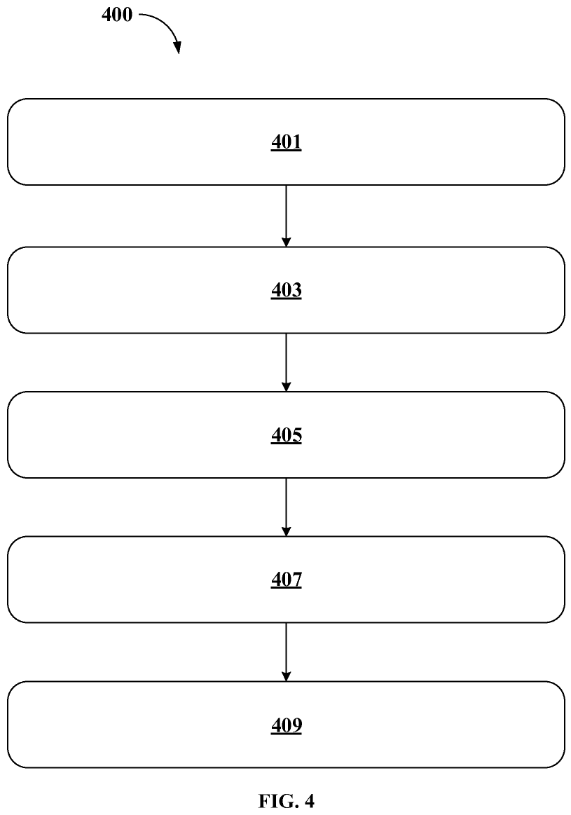

FIG. 4 shows a flowchart illustrating a method for evaluating compliance of standard clinical guidelines 107 in medical treatments of one or more patients in accordance with some embodiments of the present disclosure.

As illustrated in FIG. 4, the method 400 includes one or more blocks illustrating a method for evaluating compliance of standard clinical guidelines 107 in medical treatments of one or more patients using a health management system 105, for example the health management system 105 of FIG. 4. The method 400 may be described in the general context of computer executable instructions. Generally, computer executable instructions can include routines, programs, objects, components, data structures, procedures, modules, and functions, which perform specific functions or implement specific abstract data types.

The order in which the method 400 is described is not intended to be construed as a limitation, and any number of the described method blocks can be combined in any order to implement the method. Additionally, individual blocks may be deleted from the methods without departing from the spirit and scope of the subject matter described herein. Furthermore, the method can be implemented in any suitable hardware, software, firmware, or combination thereof.

At block 401, the method 400 comprises receiving, by the health management system 105, one or more Electronic Health Records (EHRs) 103 related to the one or more patients from one or more data sources 101. In an embodiment, the one or more EHRs 103 may be received at predetermined regular intervals. The one or more EHRs 103 may include, without limitation, information related to medical history of the one or more patients, along with the disease conditions and the socio-demographic characteristics of the one or more patients. As an example, the predetermined regular intervals may be 24 Hrs.

At block 403, the method 400 comprises aggregating, by the health management system 105, the one or more EHRs 103, corresponding to each of the one or more patients, into one or more patient-specific EHRs 211. In an embodiment, aggregating the one or more EHRs 103 may further include normalizing the information in each of the one or more EHRs 103 for eliminating one or more inconsistencies in the information stores in each of the one or more EHRs 103.

At block 405, the method 400 comprises clustering, by the health management system 105, each of the one or more patients into one or more patient groups 213 based on similarity in disease conditions and socio-demographic characteristics stored in each of the one or more patient-specific EHRs 211. As an example, the socio-demographic characteristics used for clustering each of the one or more patients may include, without limiting to, information related to the one or more patients such as age, gender, ethnicity, education level, income, location and the like.

At block 407, the method 400 comprises detecting, by the health management system 105, deviations 109 in one or more medical treatments of the one or more patients based on comparison of each of the one or more medical treatments, provided to each of the one or more patients in each of the one or more patient groups 213, with one or more standard clinical guidelines 107 associated with each of the one or more patient groups 213.

In an embodiment, the deviations 109 in the one or more medical treatments may be further classified as at least one of positive deviations and negative deviations based on impact of the deviations 109 on the disease condition and/or health condition of the one or more patients. As an example, the deviations 109 in the one or more medical treatments may be classified as the positive deviations when post-treatment health condition of the one or more patients is improved than pre-treatment health condition of the one or more patients. Similarly, the deviations 109 in the one or more medical treatments may be classified as the negative deviations when post-treatment health condition of the one or more patients is deteriorated than pre-treatment health condition of the one or more patients.

At block 409, the method 400 comprises evaluating, by the health management system 105, compliance of the one or more standard clinical guidelines 107 in the one or more medical treatments of the one or more patients based on the deviations 109 in the one or more medical treatments. In an embodiment, subsequent to evaluating the compliance of the one or more standard clinical guidelines 107, the method 400 may include recommending the one or more medical treatments that have resulted in the positive deviations to one or more practitioners for upgrading the one or more standard clinical guidelines 107. Similarly, the method 400 may further include notifying the one or more medical treatments that have resulted in the negative deviations to one or more practitioners or care takers associated with the one or more patients for facilitating the practitioners and/or caretakers in improvising the one or more medical treatments.

In an embodiment, subsequent to evaluation of the deviations 109 in the one or more treatments, each of the one or more patients may be hierarchically grouped into one or more sub-groups based on the deviations 109 in the one or more medical treatments of the one or more patients. The hierarchical groups of patients, thus formed, may be used for analyzing significance of the deviations 109 with respect to the socio-demographic characteristics of the one or more patients.

### Computer System

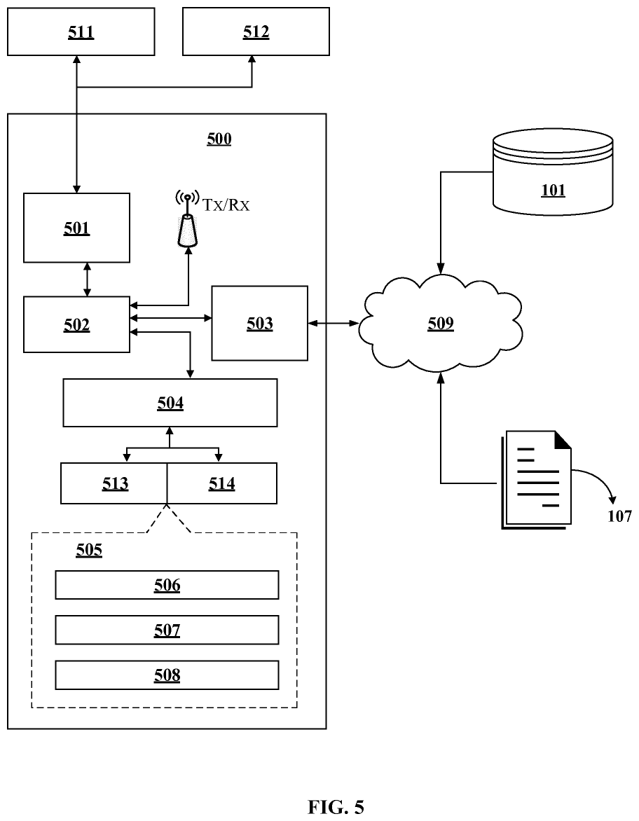

FIG. 5 illustrates a block diagram of an exemplary computer system 500 for implementing embodiments consistent with the present disclosure. In an embodiment, the computer system 500 may be health management system 105, which is used for evaluating compliance of standard clinical guidelines 107 in medical treatments of one or more patients. The computer system 500 may include a central processing unit (“CPU” or “processor”) 502. The processor 502 may comprise at least one data processor for executing program components for executing user- or system-generated business processes. A user may include a person, a patient, a practitioner and/or caretaker of the patient, a person using the health management system 105 and the like. The processor 502 may include specialized processing units such as integrated system (bus) controllers, memory management control units, floating point units, graphics processing units, digital signal processing units, etc.

The processor 502 may be disposed in communication with one or more input/output (I/O) devices (511 and 512) via I/O interface 501. The I/O interface 501 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE) or the like), etc. Using the I/O interface 501, the computer system 500 may communicate with one or more I/O devices 511 and 512.

In some embodiments, the processor 502 may be disposed in communication with a communication network 509 via a network interface 503. The network interface 503 may communicate with the communication network 509. The network interface 503 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc. Using the network interface 503 and the communication network 509, the computer system 500 may communicate with one or more data sources 101 for receiving one or more Electronic Health Records (EHRs) 103 of the one or more patients. Further, the communication network 509 may be used to retrieve one or more standard clinical guidelines 107 relevant to one or more patient groups 213 from one or more repositories, storing the one or more standard clinical guidelines 107. Furthermore, the communication network 509 may be used to communicate with an end-user device associated with the practitioner or the caretaker of the patient for communication the deviations 109 in the one or more medical treatments.

The communication network 509 can be implemented as one of the several types of networks, such as intranet or Local Area Network (LAN) and such within the organization. The communication network 509 may either be a dedicated network or a shared network, which represents an association of several types of networks that use a variety of protocols, for example, Hypertext Transfer Protocol (HTTP), Transmission Control Protocol/Internet Protocol (TCP/IP), Wireless Application Protocol (WAP), etc., to communicate with each other. Further, the communication network 509 may include a variety of network devices, including routers, bridges, servers, computing devices, storage devices, etc.

In some embodiments, the processor 502 may be disposed in communication with a memory 505 (e.g., RAM 513, ROM 514, etc. as shown in FIG. 5) via a storage interface 504. The storage interface 504 may connect to memory 505 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etc.

The memory 505 may store a collection of program or database components, including, without limitation, user/application 506, an operating system 507, a web browser 508, and the like. In some embodiments, computer system 500 may store user/application data 506, such as the data, variables, records, and the like as described in this disclosure. Such databases may be implemented as fault-tolerant, relational, scalable, secure databases such as Oracle or Sybase.

The operating system 507 may facilitate resource management and operation of the computer system 500. Examples of operating systems include, without limitation, Apple Macintosh OS X, UNIX, Unix-like system distributions (e.g., Berkeley Software Distribution (BSD), FreeBSD, Net BSD, Open BSD, etc.), Linux distributions (e.g., Red Hat, Ubuntu, K-Ubuntu, etc.), International Business Machines (IBM) OS/2, Microsoft Windows (XP, Vista/7/8, etc.), Apple iOS, Google Android, Blackberry Operating System (OS), or the like.

A user interface may facilitate display, execution, interaction, manipulation, or operation of program components through textual or graphical facilities. For example, user interfaces may provide computer interaction interface elements on a display system operatively connected to the computer system 500, such as cursors, icons, check boxes, menus, windows, widgets, etc. Graphical User Interfaces (GUIs) may be employed, including, without limitation, Apple Macintosh operating systems' Aqua, IBM OS/2, Microsoft Windows (e.g., Aero, Metro, etc.), Unix X-Windows, web interface libraries (e.g., ActiveX, Java, JavaScript, AJAX, HTML, Adobe Flash, etc.), or the like.

Furthermore, one or more computer-readable storage media may be utilized in implementing embodiments consistent with the present invention. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, nonvolatile memory, hard drives, Compact Disc (CD) ROMs, Digital Video Disc (DVDs), flash drives, disks, and any other known physical storage media.

### Advantages of the Embodiment of the Present Disclosure are Illustrated Herein

In an embodiment, the present disclosure discloses a method for retrospective evaluation of compliance of the one or more medical treatments provided to one or more patients with respect to standard clinical guidelines.

In an embodiment, the method of present disclosure helps in determining care gaps among the one or more medical treatments provided to the one or more patients, and thereby helps in providing improvised and/or patient-specific medical assistance to the patients.

In an embodiment, the method of present disclosure helps in understanding significance of availability of multiple standard clinical guidelines for a particular disease across multiple geographic regions, based on socio-demographic characteristics of a patient.

In an embodiment, the method of present disclosure helps in recommending specific amendments and/or improvements to the one or more standard clinical guidelines by detecting nature of deviations in the one or more medical treatments given to the patients.

In an embodiment, the method of present disclosure promotes using of a common standard/format such as patient-specific electronic records, for achieving interoperability among health data of the patients.

The terms “an embodiment”, “embodiment”, “embodiments”, “the embodiment”, “the embodiments”, “one or more embodiments”, “some embodiments”, and “one embodiment” mean “one or more (but not all) embodiments of the invention(s)” unless expressly specified otherwise.

The terms “including”, “comprising”, “having” and variations thereof mean “including but not limited to”, unless expressly specified otherwise. The enumerated listing of items does not imply that any or all the items are mutually exclusive, unless expressly specified otherwise.

The terms “a”, “an” and “the” mean “one or more”, unless expressly specified otherwise. A description of an embodiment with several components in communication with each other does not imply that all such components are required. On the contrary, a variety of optional components are described to illustrate the wide variety of possible embodiments of the invention.

When a single device or article is described herein, it will be clear that more than one device/article (whether they cooperate) may be used in place of a single device/article. Similarly, where more than one device or article is described herein (whether they cooperate), it will be clear that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### REFERRAL NUMERALS

| Reference Number | Description |
|---|---|
| 100 | Environment |
| 101 | Data sources |
| 103 | Electronic Health Records (EHRs) |
| 105 | Health management system |
| 107 | Standard clinical guidelines |
| 109 | Deviations in medical treatments |
| 201 | I/O interface |
| 203 | Processor |
| 205 | Memory |
| 207 | Data |
| 209 | Modules |
| 211 | Patient-specific EHRs |
| 213 | Patient groups |
| 215 | Recommendations |
| 217 | Other data |
| 219 | Receiving module |
| 221 | Data aggregation module |
| 223 | Clustering module |
| 225 | Deviation detection module |
| 227 | Deviation evaluation module |
| 229 | Other modules |
| 501 | I/O Interface of the exemplary computer system |
| 502 | Processor of the exemplary computer system |
| 503 | Network interface |
| 504 | Storage interface |
| 505 | Memory of the exemplary computer system |
| 506 | User/Application |
| 507 | Operating system |
| 508 | Web browser |
| 509 | Communication network |
| 511 | Input devices |
| 512 | Output devices |
| 513 | RAM |
| 514 | ROM |

1. A method for evaluating compliance of standard clinical guidelines in medical treatments of one or more patients, the method comprising:
receiving, by a health management system, one or more Electronic Health Records (EHRs) related to the one or more patients from one or more data sources;
aggregating, by the health management system, the one or more EHRs, corresponding to each of the one or more patients, into one or more patient-specific EHRs;
clustering, by the health management system, each of the one or more patients into one or more patient groups based on similarity in disease conditions and socio-demographic characteristics stored in each of the one or more patient-specific EHRs;
detecting, by the health management system, deviations in one or more medical treatments of the one or more patients, based on comparison of each of the one or more medical treatments, provided to each of the one or more patients in each of the one or more patient groups, with one or more standard clinical guidelines associated with each of the one or more patient groups; and
evaluating, by the health management system, compliance of the one or more standard clinical guidelines in the one or more medical treatments of the one or more patients based on the deviations, thus detected.

2. The method as claimed in claim 1, wherein the one or more EHRs are received at predetermined regular intervals.

3. The method as claimed in claim 1, wherein the one or more EHRs comprises information related to medical history of the one or more patients, along with the disease conditions and the socio-demographic characteristics of the one or more patients.

4. The method as claimed in claim 1, wherein aggregating the one or more EHRs further comprises normalizing information in each of the one or more EHRs.

5. The method as claimed in claim 1, wherein evaluating the compliance of the one or more standard clinical guidelines in the one or more medical treatments further comprises classifying the deviations in the one or more medical treatments as at least one of positive deviations and negative deviations.

6. The method as claimed in claim 5, wherein the deviations in the one or more medical treatments are classified as the positive deviations when post-treatment health condition of the one or more patients is improved than pre-treatment health condition of the one or more patients.

7. The method as claimed in claim 5, wherein the deviations in the one or more medical treatments are classified as the negative deviations when post-treatment health condition of the one or more patients is deteriorated than pre-treatment health condition of the one or more patients.

8. The method as claimed in claim 1 further comprises recommending the one or more medical treatments, resulting in positive deviations, to one or more practitioners for upgrading the one or more standard clinical guidelines.

9. The method as claimed in claim 1 further comprises notifying the one or more medical treatments, resulting in negative deviations, to one or more practitioners or care takers associated with the one or more patients.

10. The method as claimed in claim 1 further comprises hierarchically grouping each of the one or more patients into one or more sub-groups based on the deviations in the one or more medical treatments of the one or more patients, for analyzing significance of the deviations with respect to the socio-demographic characteristics of the one or more patients.

11. A health management system for evaluating compliance of standard clinical guidelines in medical treatments of one or more patients, the health management system comprising:
a processor;
a memory, communicatively coupled to the processor, wherein the memory stores processor-executable instructions, which on execution, cause the processor to:
receive one or more Electronic Health Records (EHRs) related to the one or more patients from one or more data sources;
aggregate the one or more EHRs, corresponding to each of the one or more patients, into one or more patient-specific EHRs;
cluster each of the one or more patients into one or more patient groups based on similarity in disease conditions and socio-demographic characteristics stored in each of the one or more patient-specific EHRs;
detect deviations in one or more medical treatments of the one or more patients, based on comparison of each of the one or more medical treatments, provided to each of the one or more patients in each of the one or more patient groups, with one or more standard clinical guidelines associated with each of the one or more patient groups; and
evaluate compliance of the one or more standard clinical guidelines in the one or more medical treatments of the one or more patients based on the deviations, thus detected.

12. The health management system as claimed in claim 11, wherein the processor is configured to receive the one or more EHRs at predetermined regular intervals.

13. The health management system as claimed in claim 11, wherein the one or more EHRs comprises information related to medical history of the one or more patients, along with the disease conditions and the socio-demographic characteristics of the one or more patients.

14. The health management system as claimed in claim 11, wherein the processor is configured to normalize information in each of the one or more EHRs during aggregation of the one or more EHRs.

15. The health management system as claimed in claim 11, wherein the processor is further configured to classify the deviations in the one or more medical treatments as at least one of positive deviations and negative deviations, to evaluate the compliance of the one or more standard clinical guidelines in the one or more medical treatments.

16. The health management system as claimed in claim 15, wherein the processor classifies the deviations in the one or more medical treatments as positive deviations when post-treatment health condition of the one or more patients is improved than pre-treatment health condition of the one or more patients.

17. The health management system as claimed in claim 15, wherein the processor classifies the deviations in the one or more medical treatments as the negative deviations when post-treatment health condition of the one or more patients is deteriorated than pre-treatment health condition of the one or more patients.

18. The health management system as claimed in claim 11 , wherein the processor is further configured to recommend the one or more medical treatments, resulting in positive deviations, to one or more practitioners to upgrade the one or more standard clinical guidelines.

19. The health management system as claimed in claim 11, wherein the processor is further configured to notify the one or more medical treatments, resulting in negative deviations, to one or more practitioners or care takers associated with the one or more patients.

20. The health management system as claimed in claim 11, wherein the processor is further configured to hierarchically group each of the one or more patients into one or more sub-groups based on the deviations in the one or more medical treatments of the one or more patients, to analyze significance of the deviations with respect to the socio-demographic characteristics of the one or more patients.
